(19) United States
(12) Patent Application Publication
(10) Pub. No.: US 2005/0080487 A1
(43) Pub. Date: Apr. 14, 2005

(54) Intervertebral implant

(76) Inventors: Robert Schultz, Tuttlingen (DE); Frank Altermann, Tuttlingen (DE)

Correspondence Address: RATNERPRESTIA, P O BOX 980, VALLEY FORGE, PA, 19482-0980, US

(21) Appl. No.: 10/879,449

(22) Filed: Jun. 29, 2004

(30) Foreign Application Priority Data
Dec. 12, 2003 (DE) 203 15 613.7
Oct. 8, 2003 (DE) 103 47 175.8

(51) Int. Cl. A61F 2/44

(52) U.S. Cl. 623/17.13; 623/17.14

(57) ABSTRACT

An intervertebral implant is provided for insertion between first and second vertebral bodies defining an intervertebral space. The intervertebral implant includes a first anchoring part for anchoring the intervertebral implant at the first vertebral body, a second anchoring part for anchoring the intervertebral implant at the second vertebral body, and a joint connecting the first and second anchoring parts. Joint elements of a first joint part are configured to mesh with respective ones of intermediate spaces of a second joint part and joint elements of the second joint part are configured to mesh with respective ones of intermediate spaces of the first joint part upon insertion of the intervertebral implant into the intervertebral space, thereby facilitating the tilting of the first and second anchoring parts in relation to one another.

This application is related to and claims the benefit of German Utility Model No. 203 15 613.7 entitled Intervertebral Implant issued on Dec. 12, 2003, and German Patent Application No. 103 47 175.8 filed Oct. 8, 2003.

### FIELD OF THE INVENTION

The present invention pertains to an intervertebral implant, with which the original height of the intervertebral disk can be restored, e.g., in degeneratively altered intervertebral disks, and the function is preserved at the same time.

### BACKGROUND OF THE INVENTION

Intervertebral implants are used as artificial intervertebral disk prostheses in intervertebral spaces of the human spinal column and are known in numerous embodiment variants. Numerous artificial intervertebral disk prostheses are based on the ball and socket joint principle.

For example, an intervertebral disk prosthesis with a ball and socket joint, which has a rotation center that can be fixed in relation to the anchoring parts, is known from WO 01/01893.

In order to make intervertebral disk prostheses correspond to the model of the natural intervertebral disk, protheses have been proposed whose rotation center is located in a fixed manner at one site, but can migrate during different flexing movements of the spinal column. One example of such a prior-art intervertebral disk prosthesis is described in FR 2 730 159, which comprises two metallic end plates and an intermediate part made of plastic, which has a convex bearing surface and can slide in a concave bearing surface of one of the two metal plates. Depending on where the two spherical bearing surfaces are arranged, the rotation center is located either centrally in the middle between the front or rear edge of one of the two anchoring parts or at least along a symmetry line thereof.

However, a mobility that is identical to the natural mobility is not yet reached in any of the prior-art intervertebral disk prostheses. Another problem in prior-art intervertebral disk prostheses is the wear on the joint surfaces, which happens regardless of whether these are made of plastic or metal.

Accordingly, there remains a need to improve an intervertebral implant of the type described that the most natural mobility possible of the spinal column can be restored.

### SUMMARY OF THE INVENTION

The present invention pertains to an intervertebral implant for insertion between first and second vertebral bodies defining an intervertebral space, with a first anchoring part for anchoring at the first vertebral body, with a second anchoring part for anchoring at the second vertebral body and with a joint, which connects the first and second anchoring parts and which comprises a first joint part and a second joint part, wherein the first anchoring part carries the first joint part and the second anchoring part carries the second joint part. The first joint part comprises a plurality of joint elements projecting in the direction of the second anchoring part, the second joint part comprises a plurality of joint elements projecting in the direction of the first anchoring part, and the joint elements of both joint parts are arranged and designed such that after the insertion of the intervertebral implant into the intervertebral space, they mesh with intermediate spaces between joint elements of the other joint part and make possible the tilting of the two anchoring parts in relation to one another.

Due to this design, the two joint parts can be plugged easily into one another. Moreover, especially good hold of the two joint parts in relation to one another is achieved due to the joint elements meshing with one another. Furthermore, an intervertebral implant designed in this manner makes it possible that a joint center defined by the two joint parts, for example, a rotation center, is not located in a defined position in relation to the two anchoring parts. Instead, such an intervertebral implant can migrate in the intervertebral space, exactly as it happens in the natural intervertebral disk. A translational motion within the intervertebral space of a joint center is possible depending on the design of the joint elements and the size of the intermediate spaces. Moreover, the number of support points is determined by the number of the projecting joint elements, which in turn leads to a reduction of the wear of the implant.

It is favorable if the joint elements comprise projections projecting perpendicularly or substantially perpendicularly from the anchoring parts. Such joint elements can have an especially simple design. Furthermore, their properties can be set very easily in the desired manner, for example, by selecting a special shape or by selecting certain materials.

An especially stable connection is obtained if the joint elements of the first and/or second joint part are oriented in parallel to one another. In addition, a desired displacement of the joint center within the intervertebral space can thus be set in a simple manner.

One embodiment of the invention is obtained if the joint elements of the first and/or second joint part have a bristle-like design. The two joint parts can thus be plugged into one another in a simple manner and are held at one another by the joint elements of the other joint part immersing into the intermediate spaces of the respective other joint part.

Provisions may be made according to a preferred embodiment of the present invention for the intermediate spaces between joint elements of one of the two joint parts to be arranged and designed such that they receive the joint elements of the respective other joint part and make possible only a relative movement of the two anchoring parts toward or away from one another. This means that the intermediate spaces between joint elements of one joint part are designed such that the joint elements of the other joint part fit exactly in between these, i.e., no translational motion would be possible in a direction at right angles to a connection direction between the two joint parts if the joint elements were inelastic or nonarticulated. A translational motion of the joint center of the intervertebral implant can then be embodied by the special design of the joint parts, for example, by the length, mounting, or elasticity properties of these joint parts.

The joint elements of the first joint part are preferably all of equal length. Free ends of the joint elements thus define a first, flat joint surface.

It is advantageous if the joint elements of the second joint part define a hemisphere with their free ends. A spherical joint part can be formed in this manner. If, for example, all the joint elements of the other joint part are shorter, an anchoring surface of the joint elements of the other joint part can roll on the free ends of the joint elements defining a hemisphere. A ball and socket joint can thus be formed in a simple manner. In addition to a pure rotation, a special design of the joint elements, for example, based on the elasticity of these joints elements, can additionally make possible a translational motion within the intervertebral space for the joint center.

It is advantageous if the joint elements of the first joint part are shorter than the longest joint elements of the second joint part. Free ends of the joint elements of the second joint part thus form stops for a bearing surface of the joint elements of the first joint part. A ball and socket joint can thus be formed in a simple manner, for example, in case of joint elements of equal length of the first joint part and free ends of the second joint part that define a hemisphere.

In order to also make possible a translational motion of the joint center of the intervertebral implant in addition to a rolling movement, the joint elements of the first and/or second joint part may be elastic. This makes possible the bending of the joint parts out of a normal position as well as compression or elongation of the same joint parts. Due to their elastic design, the joint elements return to their original shape especially easily.

In order to increase the mobility of the joint elements, provisions may be made for the joint elements of the first and/or second joint part to be mounted at a respective anchoring part in an articulated manner. Thus, joint elements can be mounted, for example, pivotably at the respective anchoring part, resulting in that a translational motion of the joint center can be preset in the desired manner.

It is especially advantageous if ball and socket joints are provided for mounting the joint elements of the first and/or second joint part. The joint elements can be pivoted by means of ball and socket joints in all three directions of space, so that a joint center of the joint elements can also migrate, in principle, in all directions within the intervertebral space.

To prevent damage to the intervertebral implant and to limit a tilting movement of vertebral bodies of the spinal column that are connected via the intervertebral implant, it may be favorable for the ball and socket joints to permit a pivoting movement by a maximum of 40° starting from a normal position that is perpendicular in relation to the corresponding anchoring part.

It would be conceivable, in principle, to arrange the joint elements directly at the anchoring part and to rigidly connect them with the anchoring part. However, it is favorable if the first joint part can be connected with the first anchoring part and/or the second joint part can be connected with the second anchoring part. This makes it possible to replace joint parts should this become necessary. Moreover, taking into account special anatomic situations, an optimal joint part can be selected in conjunction with preferred anchoring parts.

The design and especially the replacement of joint parts is facilitated if at least one of the two joint parts comprises a joint element carrier, which carries at least some of the majority of joint elements of the at least one joint part. For example, this joint element carrier could also carry all joint elements of the joint part.

The at least one joint element carrier can be preferably connected with one of the two anchoring parts. This embodiment makes it possible that joint parts can be replaced in a simple manner, e.g., for separating the joint element carrier from the anchoring part. In addition, a set of different joint parts can be combined as a result with an anchoring part or with different anchoring parts in order to make possible the optimal reconstruction of a natural intervertebral disk.

To ensure an especially good hold of the joint parts at one or both of the anchoring parts, it is advantageous if at least one of the two anchoring parts has a joint part mount for receiving one of the two joint parts. For example, the joint part mount could be designed in the form of a depression in a lateral face or surface of the anchoring part.

In order to make it possible to establish the connection between the joint part and the anchoring part securely and reliably, it is advantageous if the at least one joint element carrier can be connected with one of the two anchoring parts in a nonpositive and/or positive-locking manner. For example, a joint element carrier can be inserted into a joint element mount in a positive-locking manner. In addition, grooves with undercuts can guarantee a secure hold against the lifting off of the joint element carrier from the anchoring part. It would also be conceivable to fix the joint element carrier at the anchoring part by means of a press fit. However, it would also be possible to insert the joint element carrier into a joint element mount in a positive-locking manner only. In this positive-locking case, the cross sections of the joint element mount and of the joint element carrier can be designed such that rotation around a perpendicular axis or an axis projecting from the anchoring part substantially perpendicularly is possible.

Provisions may be made according to a preferred embodiment of the present invention to provide a set of different joint parts with joint element carriers of different thicknesses and/or with different joint elements, which have a different thickness and length in the direction of the respective other anchoring part. Natural distances between adjacent vertebral bodies can thus be reconstructed with different joint parts. Depending on the patient's size and corresponding to the original distances of the vertebral bodies, correspondingly optimized joint parts can be selected and inserted into the intervertebral space.

To set the movement of the joint in the desired manner, the joint elements of the first and/or second joint part may have different elasticities. For example, the joint elements of one joint part may be completely rigid, and those of the other joint part may be elastic. It would also be conceivable to make individual joint elements of a joint part rigid and others elastic. Furthermore, it is also conceivable that joint elements of one joint part have different elasticities, for example, joint elements that are arranged farther in the center of the joint part may be made less elastic and joint elements arranged farther at the edge of the joint part may be made more elastic.

To embody different elasticities in a simple manner, the joint elements of the first and/or second joint part may be made, for example, from different materials. It would also be conceivable to manufacture joint elements of one joint part from different materials in order to make possible the above-described different elasticities.

The joint elements of the first and/or second joint part preferably have different cross sections. The elasticities of the joint elements can thus be set in an especially simple manner.

To define the elasticity of a joint part in the desired manner, it may be favorable to provide joint elements that have a cross section changing in the direction of the other anchoring element. Rolling movements of the two anchoring parts in relation to one another can be predetermined as a result in the desired manner in a simple fashion.

The cross section of at least one of the joint elements preferably increases in the direction of the other joint surface. A secure hold of the two joint parts at one another is thus guaranteed, especially during a rolling movement of the two joint parts in relation to one another. The joint elements, which are, for example, elastic, are additionally widened during a rolling movement, so that joint elements with a cross section increasing in the direction of the other joint surface ensure the especially favorable filling of intermediate spaces between the joint elements of the other joint part. A result is that the friction and consequently the wear of the joint parts are also minimized.

In order to achieve the most optimal movement of the joint possible and to minimize friction, it may be advantageous for the joint elements to have a free end of a hemispherical shape.

The design of the intervertebral implant becomes especially simple if the joint elements have a round or substantially round cross section. The two joint parts can thus be plugged into one another, and the joint elements form the densest bar packing, i.e., they mesh with one another such that no movement would be possible at right angles to the longitudinal direction of the joint elements in the case of rigid joint elements.

To make it possible to preset a relative movement of the two anchoring parts in the desired manner, it is advantageous if the joint elements of the first and/or second joint part are arranged in a grid-like pattern. Desired movement limitations can thus be set in a simple manner, for example, tilt angles of the two anchoring parts in relation to one another or limits of a possible translational motion within the intervertebral space for the joint center.

An especially simple design of the intervertebral implant is obtained if the joint elements are arranged in a square grid.

To ensure the densest possible and optimal meshing of joint elements of the joint parts with one another, it is favorable if two adjacent joint elements of the first joint part are located at a distance from one another that corresponds maximally to a diameter of the joint elements of the second joint part. The densest packing of the mutually meshing joint parts can thus be formed.

It would be possible, in principle, to fix the intervertebral implant on surfaces of natural vertebral bodies. However, provisions may be made according to a preferred embodiment of the present invention for the first and/or second anchoring parts to be able to be connected with a vertebral body replacement implant. The case in which vertebral bodies must be completely or partially replaced with vertebral body replacement implants may occur in degenerative diseases of the spinal column. The special design of the intervertebral implant now makes it possible to also connect the implant directly with a vertebral body replacement implant, so that conventional, commercially available vertebral body replacement implants can be connected with the intervertebral implant according to the present invention.

However, it is also conceivable that the first and/or second anchoring part is connected with a vertebral body replacement implant. For example, such an anchoring part can be made integrally in one piece with the vertebral body replacement implant, especially making them in one piece.

The joint elements may be made, in principle, from any desired material. However, they are preferably manufactured from a physiologically compatible material.

Depending on the use or the desired elasticity of the joint elements, it is advantageous if the physiologically compatible material is a metal or a plastic. In particular, joint elements from fiber-reinforced plastic are conceivable. As an alternative, the joint elements may be manufactured from a titanium alloy. The at least one joint element carrier is preferably manufactured from a metal, especially a chromium-cobalt alloy. However, joint element carriers from a titanium alloy or plastic would be conceivable as well.

### BRIEF DESCRIPTION OF THE DRAWINGS

FIG. 1 shows a first variant of an intervertebral disk inserted into an intermediate space between two vertebral bodies;

FIG. 2 shows a sectional view along line 2-2 in FIG. 1;

FIG. 3 shows a perspective view of a first anchoring part of the intervertebral disk prosthesis from FIG. 1;

FIG. 4 shows a perspective view of a second anchoring part of the intervertebral disk prosthesis from FIG. 1; and

FIG. 5 shows a cross-sectional view of a second exemplary embodiment of the intervertebral disk prosthesis inserted into an intervertebral space between two vertebral bodies.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

FIG. 1 shows an intervertebral disk prosthesis designated as a whole by the reference number 10. It is inserted into an intervertebral space 12 between a first vertebral body 14 and a second vertebral body 16.

The intervertebral disk prosthesis 10 has a two-part design and comprises a first carrier plate 18 and a second carrier plate 20, which comprise a respective anchoring surface 22 and 24 and a respective bearing surface 26 and 28. Narrow, plate-like anchoring ribs 30 and 32, which are driven into the vertebral bodies 14 and 16, respectively, to anchor the carrier plates 18 and 20 or are inserted into recesses 34 and 36 prepared therein for this purpose, project perpendicularly from the anchoring surfaces 22 and 24. The anchoring surfaces 22 and 24 now lie substantially flat on respective surfaces 38 and 40 of the respective vertebral bodies 14 and 16, which point toward one another and define the intervertebral space 12 between them. The shape of the anchoring surfaces 22 and 24 substantially corresponds to the surfaces 38 and 40 of the vertebral bodies 14 and 16, so that the largest possible coverage is achieved between the anchoring surfaces 22 and 24 and the surfaces 38 and 40.

The material of the carrier plates 18 and 20 is preferably a biocompatible metal such as, e.g., a titanium alloy or a chromium-cobalt alloy.

The intervertebral disk prosthesis 10 comprises, furthermore, a joint 42, which is formed by a first joint part 44 connected with the carrier plate 18 and a second joint part 46 connected with the carrier plate 20.

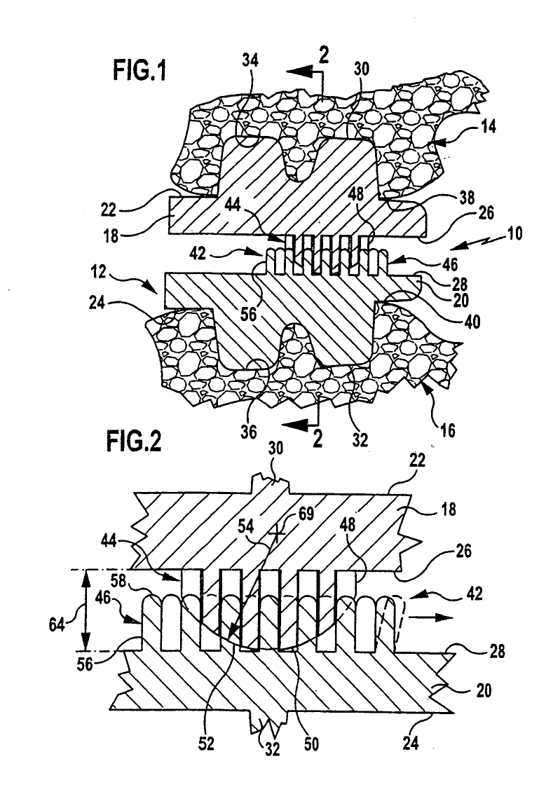

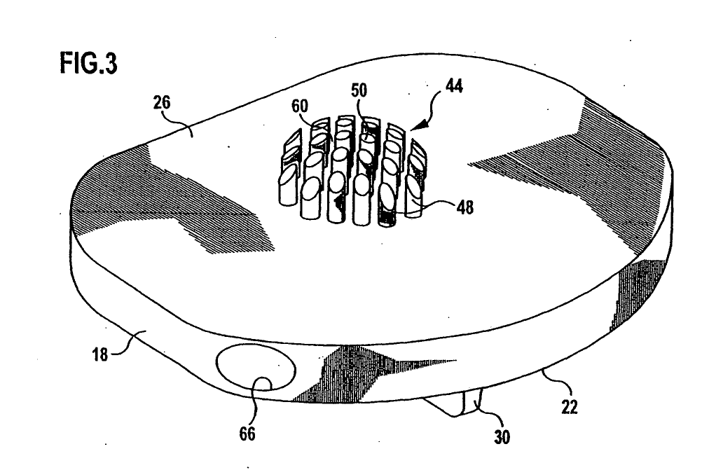

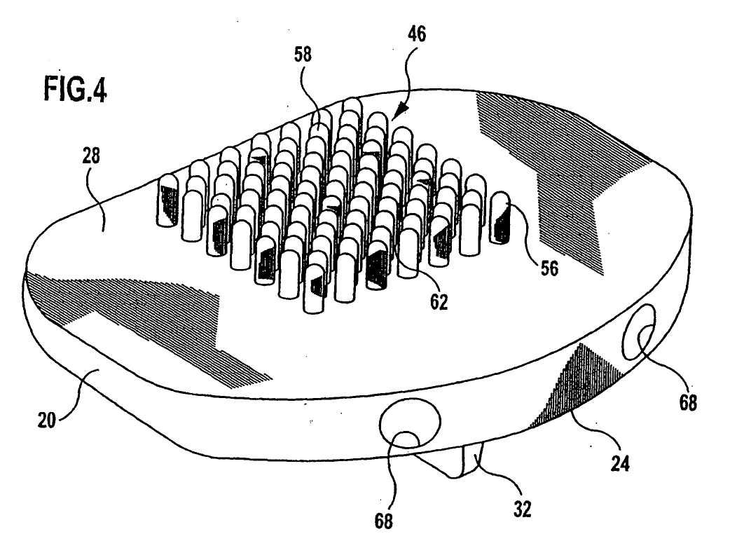

The design of the joint parts 44 and 46 can be recognized especially well from FIGS. 3 and 4, respectively. Referring to FIG. 3, the joint part 44 comprises a plurality of joint bristles 48 projecting perpendicularly from the bearing surface 26. These bristles 48 have a circular cross section and are arranged at intersections of a square grid on the bearing surface 26. All the joint bristles 48 of the first joint part 44 have essentially different lengths, so that free bristle ends 50 of the joint bristles 48 define points of a spherical surface 52, which has the radius 54 (represented in FIG. 2).

Referring to FIG. 4, a plurality of identical support bristles 56 of round cross section project perpendicularly from the bearing surface 28 of the carrier plate 20. The free support bristle ends 58 of the support bristles 56 are hemispherically round. Similar to the bristle ends 50 of the joint bristles 48, the support bristles 56 are likewise arranged on the bearing surface 28 at intersections of a square grid. The support bristles 56 thus cover a square partial area of the bearing surface 28. The grids of the joint bristles 48 and of the support bristles 56 are selected to be such that exactly one support bristle 56 of the second joint part 46 fits an intermediate space 60 of the first joint part 44, which intermediate space 60 is surrounded by four joint bristles 48. Conversely, exactly one joint bristle 48 fits an intermediate space 62 of the second joint part 46 that is surrounded by four support bristles 56. It is possible in this manner to plug the first joint part 44 into the second joint part 46 or vice versa. FIGS. 1 and 2 show the intervertebral disk prosthesis 10 in an assembled position, in which all joint bristles 48 immerse into intermediate spaces 62 between the support bristles 56.

The minimum distance between the bearing surfaces 26 and 28 of the two carrier plates 18 and 20 is defined by the length of the longest joint bristles 48, as represented in FIG. 2 as distance 64. All support bristles 56 are shorter than the longest joint bristles 48. As a result, free bristle ends 50 of the first joint part 44 directly abut against the bearing surface 28 of the second carrier plate 20. The first carrier plate 18 comprises two blind holes 66 (one hole 66 shown in FIG. 3), which extend in parallel to the bearing surface 26 and act as a tool mount for an inserting instrument for inserting the intervertebral disk prosthesis 10 into the intervertebral space 12. Two blind holes 68 (shown in FIG. 4), which are mutually parallel to the bearing surface 28 and the anchoring rib 32 and act as instrument mounts, are provided in a similar manner in second carrier plate 20.

The joint bristles 48 and the support bristles 56 are each firmly connected with the bearing surfaces 26 and 28, respectively. For example, the joint bristles 48 and the support bristles 56 may be bonded to the bearing surfaces 26 and 28, respectively. Alternatively, the joint bristles 48 and the support bristles 56 may be screwed, for example, by providing a threaded section, which can be screwed into corresponding threaded holes of the carrier plates 18 and 20, respectively. It would also be possible to provide the carrier plates 18 and 20 in one piece with joint bristles 48 and support bristles 56.

The joint bristles 48 and the support bristles 56 are made from an elastic material, so that they can bend away from their normal position in which they project perpendicularly from the bearing surfaces 26 and 28, respectively. A support bristle 56 in a deflected position is represented by a broken line in FIG. 2. The joint part 44 can thus quasi roll on the bearing surface 28. At the same time, the elastic joint bristles 48 and support bristles 56 make it possible that a rotation center 69 of the joint 42 is not always located in the same position in relation to the carrier plate 18, but can migrate in all directions within the intervertebral space 12. The rolling movement is a combination of rotation and a superimposed translational motion. A movement characteristic that corresponds substantially to the model of the natural intervertebral disk is thus obtained for the intervertebral disk prosthesis 10. There are no sliding surfaces, and therefore the amount of wear is nearly equal to zero.

The radius 54 of the joint bristles 48 defines the extent of the translational motion. This radius 54 is selected to be such that the translational motion remains relatively small. Even though such translational motions do occur in the movement pattern of the natural intervertebral disk and are desirable, they are not forced by the intervertebral disk itself, but are affected by the surrounding structures such as ligaments, muscles, and facet joints. It is therefore desirable that the principal translational motion is made possible by the elasticity of the bristles 48 and 56 and becomes established passively in response to the forces acting from the outside.

Bristles 48 and 56 of different elasticities may be utilized. The extent of movement of the intervertebral disk prosthesis 10 can thus be additionally affected, so that a prosthesis 10 can be equipped with more or less elastic bristles 48 and 56 depending on the desired degree of stabilization.

The different elasticities of the bristles 48 and 56 can be brought about either by means of materials of different hardness or by affecting the elasticity of the bristles 48 and 56 by design measures (e.g., different diameters), even though the bristles 48 and 56 are made of the same material.

The bristles 48 and 56 may be made of a physiologically compatible, metallic material. Alternatively, the bristles 48 and 56 may be made from homogeneous plastics or fiber-reinforced plastics.

The position of the joint parts 44 and 46 on the bearing surfaces 26 and 28 may be selected essentially as desired, i.e., respective joint parts 44 and 46 may be arranged centrally on the respective bearing surfaces 26 and 28 or they may be offset anteriorly or posteriorly, as indicated in FIGS. 1 through 4.

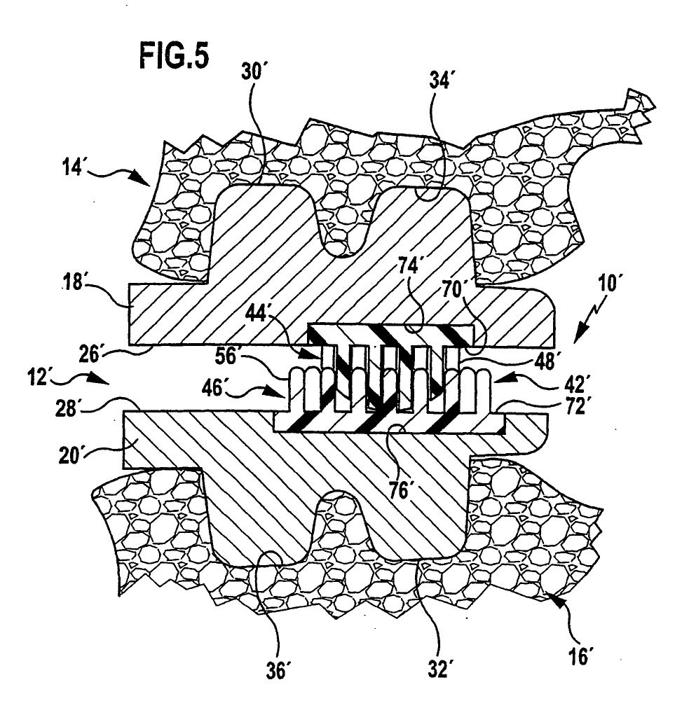

A second exemplary embodiment of an intervertebral disk prosthesis, which is designated as a whole by the reference number 10′, is shown in FIG. 5. Its basic design corresponds to the intervertebral disk prosthesis 10 as it was explained above in greater detail with reference to FIGS. 1 through 4. However, it differs from the intervertebral disk prosthesis 10 in that the joint parts 44′ and 46′ of joint 42′ are not arranged directly on respective bearing surfaces 26′ and 28′ of the respective carrier plates 18′ and 20′. Instead, joint bristles 48′ forming the joint part 44′ are arranged on an inlay plate 70′, and the support bristles 56′ forming the joint part 46′ are arranged on an inlay plate 72′. The inlay plate 70′ has a disk-shaped design and is inserted in a positive-locking manner into a round depression 74′ of the carrier plate 18′, which points in the direction of the carrier plate 20′. A square depression 76′ is provided in a similar manner in the bearing surface 28′ of the carrier plate 20′, which receives the square inlay plate 72′ carrying the support bristles 56′ in a positive-locking manner. Such a configuration facilitates replacement of joint parts 44′ and 46′ as desired. Inlay plates 70′ and 72′ with different heights may be selected to achieve favorable adaptation to the particular height of the intervertebral disk space 12′.

The inlay plates 70′ and 72′ are made of a biocompatible material, preferably a metal, especially a titanium alloy or a chromium-cobalt alloy. The joint bristles 48′ and the support bristles 56′ may be made in one piece with the respective inlay plates 70′ and 72′. It would also be conceivable, as was described above, to make the joint bristles 48′ and the support bristles 56′ from a material that is different from the material of the respective inlay plates 70′ and 72′ and to connect the two types of bristles with one another. The joint bristles 48′ and the support bristles 56′ may be made from the same materials as the joint bristles 48 and the support bristles 56 described above with reference to FIGS. 1 through 4.

In use, the intervertebral disk prosthesis 10 and 10′ is implanted in its completely assembled state, thereby significantly simplifying the implantation procedure.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. An intervertebral implant for insertion between first and second vertebral bodies defining an intervertebral space, said intervertebral implant comprising:
a first anchoring part for anchoring said intervertebral implant at the first vertebral body;
a second anchoring part for anchoring said intervertebral implant at the second vertebral body; and
a joint connecting said first and second anchoring parts, said joint comprising:
a first joint part carried by said first anchoring part, said first joint part comprising a plurality of joint elements projecting in a direction toward said second anchoring part, said joint elements defining intermediate spaces between said joint elements, and
a second joint part carried by said second anchoring part, said second joint part comprising a plurality of joint elements projecting in a direction toward said first anchoring part, said joint elements defining intermediate spaces between said joint elements,
wherein said joint elements of said first joint part are configured to mesh with respective ones of said intermediate spaces of said second joint part and said joint elements of said second joint part are configured to mesh with respective ones of said intermediate spaces of said first joint part upon insertion of said intervertebral implant into the intervertebral space, thereby facilitating the tilting of said first and second anchoring parts in relation to one another.

2. The implant of claim 1, wherein said joint elements of said first and second joint parts comprise projections projecting substantially perpendicularly from said anchoring parts.

3. The implant of claim 1, wherein said joint elements of said first and/or second joint part are oriented in parallel to one another.

4. The implant of claim 1, wherein said joint elements of said first and/or second joint part have a bristle-like design.

5. The implant of claim 1, wherein said intermediate spaces of said first and second joint parts permit only a relative movement of said first and second anchoring parts toward one another or away from one another.

6. The implant of claim 1, wherein all of said joint elements of said first joint part are of equal length.

7. The implant of claim 1, wherein said joint elements of said second joint part comprise free ends of varying lengths defining a hemispheric shape.

8. The implant of claim 7, wherein said joint elements of said first joint part are shorter than said longest joint elements of said second joint part.

9. The implant of claim 1, wherein said joint elements of the said first and/or second joint part are elastic.

10. The implant of claim 1, wherein said joint elements of said first and/or second joint part are mounted at said respective anchoring part in an articulated manner.

11. The implant of claim 10 further comprising ball and socket joints for mounting said joint elements of said first and/or second joint part.

12. The implant of claim 11, wherein said ball and socket joints permit a pivoting movement by a maximum of 40° starting from a normal position that is perpendicular in relation to said respective anchoring part.

13. The implant of claim 1, wherein said first joint part can be connected with said first anchoring part and/or said second joint part can be connected with said second anchoring part.

14. The implant of claim 1, wherein at least one of said first and second joint parts comprises a joint element carrier configured to carry at least some of a majority of said joint elements of said at least one joint part.

15. The implant in accordance with claim 14, wherein said joint element carrier can be connected with one of said first and second anchoring parts.

16. The implant of claim 1, wherein at least one of said first and second anchoring parts has a joint part mount for receiving one of said first and second joint parts.

17. The implant of claim 14, wherein said joint element carrier can be connected with one of said first and second anchoring parts in a nonpositive and/or positive-locking manner.

18. The implant of claim 14, wherein a set of said first and second joint parts comprise joint element carriers of different thicknesses and/or joint elements of different thickness and length projecting in a direction of said respective other anchoring part.

19. The implant of claim 1, wherein said joint elements of said first and/or second joint part comprise different elasticities.

20. The implant of claim 1, wherein said joint elements of said first and/or second joint part are made of different materials.

21. The implant of claim 1, wherein said joint elements of said first and/or second joint part have different cross sections.

22. The implant of claim 1, wherein the cross section of said joint elements varies.

23. The implant of claim 22, wherein the cross section of at least one of said joint elements increases in a direction toward said other anchoring part.

24. The implant of claim 1, wherein said joint elements comprise free ends of hemispherical shape.

25. The implant of claim 1, wherein the cross section of said joint elements is substantially round.

26. The implant of claim 1, wherein said joint elements of said first and/or second joint part are arranged in a grid-like pattern.

27. The implant of claim 26, wherein said joint elements are arranged in a square grid.

28. The implant of claim 1, wherein adjacent joint elements of said first joint part are spaced apart from each other at a distance that corresponds maximally to the diameter of said joint elements of said second joint part.

29. The implant of claim 1, wherein said first and/or second anchoring part is configured to be connected with a vertebral body replacement implant.

30. The implant of claim 29, wherein said first and/or second anchoring part is connected with the vertebral body replacement implant.

31. The implant of claim 1, wherein said joint elements are made of a physiologically compatible material.

32. The implant of claim 31, wherein said physiologically compatible material is a metal or a plastic.
